(19) United States
(12) Patent Application Publication
(10) Pub. No.: US 2005/0048193 A1
(43) Pub. Date: Mar. 3, 2005

(54) Porous metals and metal coatings for implants

(75) Inventors: Jia Ping Li, Utrecht (NL); Pierre Jean F. Layrolle, Nantes (FR); Klaas De Groot, Heemstede (NL)

Correspondence Address: BANNER & WITCOFF, LTD., 28 STATE STREET, 28th FLOOR, BOSTON, MA, 02109-9601, US

(73) Assignee: ISOTIS N.V., Bilthoven (NL)

(21) Appl. No.: 10/647,022

(22) Filed: Aug. 18, 2003

Related U.S. Application Data
(63) Continuation of application No. PCT/NL02/00102, filed on Feb. 18, 2002.

(30) Foreign Application Priority Data
Feb. 19, 2001 (EP) 01200587.2
May 30, 2001 (EP) 01202062.4

(51) Int. Cl. B32B 3/12; A61L 2/00

(52) U.S. Cl. 427/2.24; 427/2.27; 623/926; 428/158

(57) ABSTRACT

The invention is directed to a method of preparing porous metals, as well as to these porous metals per se. More in particular the invention is directed to the use of these porous metals in the preparation of medical items, such as implants. The invention further relates to a method of providing a porous metal coating on a substrate, in particular on the surface of a medical item, such as an implant or scaffold for tissue engineering. According to the method of the invention, a polymeric foam is impregnated with a slurry of metal particles, such as titanium, tantalum, titanium alloy or tantalum alloy particles. The impregnated foam is subsequently dried and subjected to pyrolysis and subsequent sintering. Due to the presence of metal hydrides, the formation of undesired compounds, such as metal oxides or nitrides, is avoided.

The invention is directed to a method for preparing porous bodies, suitable for the preparation of porous metal articles, as well as to these porous metal articles per se. More in particular the invention is directed to the use of these porous metals in the preparation of medical items, such as implants or scaffolds in tissue engineering. The invention further relates to a method of providing a porous metal coating on a substrate, in particular on the surface of a medical item, such as an implant or scaffold for tissue engineering.

Because of their excellent characteristics, titanium, tantalum and alloys thereof find use in medical devices, such as implants. These materials provide good biocompatibility, are lightweight, have a high strength, and superior corrosion resistance. Great effort has been given to the application of these materials in the production of medical equipment, such as dental implants, clips for blood vessels, artificial bones, artificial joints, etc. Most of these applications use the dense phase of these metals. The use of powder metallurgy for fabrication of orthopedic joint replacement implants was first reported in the mid-1960s. Porous titanium was first used for dentistry in animals in American Medical Center of Luke and University of Chicago in 1969.

Regeneration of skeletal tissues has been recognized as a new means for reconstruction of skeletal defects arising from abnormal development, trauma, tumors and other conditions requiring surgical intervention. Autologous bone grafting is considered as the golden standard of bone transplantation with superior biological outcomes. However, autologous bone stocks are limited and often insufficient, particularly when large skeletal defects are encountered. As surgical techniques and medical knowledge continue to advance, there is an increasing demand for synthetic bone replacement materials. Variation of the scaffold design as three-dimensional superstructures has been demonstrated as an approach to optimize the functionality of bone regeneration materials so that these materials may be custom designed for specific orthopedic application in the form of void fillers, implants, or implant coating. In an attempt to develop a skeletal cell and tissue carrier, which could provide optimal spatial conditions for cell migration and maintenance by the arrangement of structural elements such as pores and fibers, the feasibility of using “live” material is under investigation. Such live material could take the form of an open-porous implant system together with living tissue. This technique is also referred to as hard tissue engineering.

Several methods are already known to make porous metals, such as titanium. Examples of these known methods are isostatic pressing (ISP) sintering, rolling sintering, loose packed sintering and fiber-wired sintering. In general, according to these known methods, titanium particle are mixed together with binders or loosely packed, and subsequently sintered. The packing of the particles then leaves a porous structure. However, the porous metals made by these known methods have shortcomings. Usually the porosity is too low, i.e. below 50%. Also the pore size is generally too small, the maximum pore size being about 300 μm.

Another method to make porous metals, such as titanium is hammer-pressing metal fiber. Although the porosity obtained by this method is above 70%, the strength is generally too low and the pore size is still too small.

For use as implants, the pore size and porosity are important for the cells to grow inside after implantation. In general, the porous metal should, apart from the above-mentioned chemical requirements of good biocompatibility, lightweight and superior corrosion resistance, meet the following requirements: the porosity should be 50% or more, the average pore size should be at least 400 μm, preferably at least 500 μm. Preferably the average pore size should not exceed 800 μm. In addition, the pores should be interconnected and the compressive strength should be sufficient for load-bearing purposes. In particular, the mechanical compressive strength of porous titanium alloy should be at least 5 MPa.

Further, U.S. Pat. No. 6,136,029 discloses a process for the preparation of ceramic porous bone substitute material. This known process is, however, not suitable for the preparation of metal articles. The pyrolysis and subsequent sintering according to this known method, will give rise to formation of undesired metal compounds, such as metal nitrides and oxides, in particular on the outer surface of the porous articles. For use as implants, the presence of these compounds, in particular on the outer surface, is not acceptable, because the formation of metal nitride or oxides will give rise to a decrease of mechanical strength. Metal nitrides or oxides such TiN or TiO₂ compounds are formed in the presence of air (N₂/O₂/H₂O) at the high temperature reached during sintering of metals (e.g. 1250° C.). Titanium is a very reactive metal and can react with nitrogen, oxygen or water to form nitride or oxide at temperature as low as 700° C. according to the following equations:
Ti+½N₂->TiN
Ti+O₂->TiO₂

Up until now, it has not been possible, or only with great difficulty, to provide porous metals, suitable for implants, which meet the above-mentioned requirements and/or do not suffer from the above-mentioned shortcomings. It is an object of the present invention to provide for a method, with provides for a substantial improvement regarding the above-mentioned requirements and drawbacks in respect to the methods of the prior art.

The present inventors have found that this object can be met by preparing porous bodies, from which metal articles can be made, by the so-called slip casting process. The slip casting process comprises the preparation of a body by the impregnation of a pyrolysable foam material, such as a polymer, with a slurry of metal particles, and subsequent pyrolysis of the foam material. This may subsequently be followed by sintering of the body. Therefore, in a first embodiment, the present invention is directed to a method for preparing a porous body, suitable for the production of a porous metal article, comprising the steps of providing a polymeric foam, which foam is impregnated with a slurry of metal particles, drying the impregnated foam, followed by pyrolysis in the presence of metal hydride particles.

Furthermore, the present invention provides a method for preparing a porous metal article comprising sintering of the body thus obtained, which sintering is carried out in the presence of metal hydride particles. A porous metal article according to the invention has a good biocompatibility, and is lightweight, combining a high strength with good corrosion resistance.

In a second embodiment, the instant invention relates to the provision of a porous metal coating onto a substrate.

U.S. Pat. No. 4,636,219 “Prosthesis device fabrication” (Techmedica Inc.) discloses a process for fabricating a biocompatible mesh screen structure for bonding to a prosthetic substrate. The method consists of applying four to eight layer of a mesh at a pressure of 1300 to 1500 psi and temperature of 1600 to 1725 F under vacuum of less than 10E-4 torr.

U.S. Pat. No. 5,443,510 “Porous coated implant and method of making same” (Zimmer Inc.) teaches a method for applying beads or wire mesh on the implant surface using conventional welding techniques, matching, bead-blasting and finally sintering.

U.S. Pat. No. 4,969,904 “Bone implant” (Sulzer) describes a method to apply a wire mesh to a metal substrate using pressure and mechanical interlock.

U.S. Pat. No. 5,507,815 “Random surface protrusions on an implantable device” (Cycam Inc.) discloses a masking-chemical etching method to provide a random irregular pattern onto surface of implantable device.

None of the methods disclosed in the above discussed prior art can provide highly porous metal coatings with a high pore interconnection for bone growth. Further, the resulting porous coating is not very well attached to the prosthesis substrate.

It has been found that the principles of the instant method for preparing a porous metal article can also be employed to provide porous coatings of metal materials to a substrate. In accordance with this embodiment, the polymeric foam impregnated with a slurry of metal particles is pasted onto the substrate to which the coating is to be applied. After sintering, a homogeneous attachment of the coating to the substrate is achieved, in particular when the coating and the substrate comprise the same metal. Hence, it is preferred that the substrate is of the same metal as the porous coating or, if the substrate is an alloy, it is preferred that said alloy comprises at least 50 wt. % of the metal of the porous coating. Although it is possible to provide alloy coatings, it is preferred that the coating is composed of one metal only.

The term “presence” as used herein with respect to metal hydride particles, is to be interpreted in its broadest sense, viz. it is sufficient to carry out the pyrolysis or the sintering in an environment, in which the metal hydride particles are also present. Preferably the metal hydride is substantially not in contact with the impregnated foam or the body. This may e.g. be effected by placing the sample to be pyrolyzed or sintered in an oven, while the metal hydride is present in a different location of the same oven. It was found that the presence of the metal hydride particles is an important aspect of the method of the present invention, since these particles prevent the formation of undesired metal compounds, such as oxides and/or nitrides (e.g. titanium oxide and/or titanium nitride). Presence of these undesired metal compounds would make the articles unsuitable for medical use, e.g. as implants. In this respect it is stressed that although the pyrolysis and subsequent sintering are usually and preferably carried out in vacuum (in practice this means pressures of about 0.5 mPa up to several Pa), the presence of reactive gases, in particular of oxygen and nitrogen from air, as well as water, can never completely be avoided, even not if these steps are carried out in an inert gas, such as argon. In addition, the slip casting method involves the impregnation of a foam material with a slurry of metal particles, as a result of which air, water and/or other contaminants may become captured in the impregnated body, which contaminants cannot be removed by e.g. lowering the pressure and/or flushing with inert gas.

As a consequence, if no countermeasures are taken, the formation of undesired metal compounds is inevitable. Presence of these undesired metal compounds is already detrimental in very low concentrations.

Without wishing to be bound by theory, it is assumed that the metal hydride particles are much more reactive with respect to contaminants, such as air and water, than the metal particles. As a result, the metal hydride particles act as a scavenger and react with these contaminants under pyrolysis or sintering conditions, so that the metal particles are protected against undesired nitruration or oxidation. In addition, the fusion of the metal particles during sintering is enhanced by the absence of the metal nitrides and oxides, resulting in an increased mechanical stability of the final article.

The metal hydride particles, which serve as a scavenger may be introduced by impregnating the foam with a slurry of these metal hydride particles. For convenience, it is preferred that the metal hydride particles are present in the same slurry as the metal particles. As was stated above, it is however preferred not to provide the metal hydride particles in a slurry in the foam, but to provide these particles separately from the impregnated foam, viz. on a different location in the same environment.

The slurry of metal particles, and optionally metal hydride particles is prepared by mixing said particles with water under stirring until a homogenous slurry is obtained. Generally, a concentration will be chosen between 50% and 80 wt. %, preferably between 55 and 75 wt. %, based on the weight of the slurry.

In order to obtain a stable slurry, the addition of a binder is preferred. The concentration of binder is an important measure for controlling the viscosity of the slurry. With the increase of the amount of the binder, the sedimentation rate of the particles decreases because of the increasing viscosity of the slurry. It has been found that the optimal viscosity ranged from 4000 (centipoises) cps to 12000 cps, if the viscosity is too high, it is difficult to remove the extra slurry after impregnation. Suitable concentrations for the binder are 2-15 wt. %, preferably 4-9 wt. %. The criteria for selecting the binder material are that the binder should not react with metal powder and that it should be removed completely after the sintering of the samples. Suitable binders are e.g. PEG4000, methylcellulose and/or carboxyl methyl cellulose (CMC), polyolefins such as polyethylene or polypropylene, ethylene vinyl acetate, styrene group resins, cellulose derivatives, various types of wax; paraffin, and the like.

Particularly suitable metal particles are made from titanium, tantalum, titanium alloy, tantalum alloy, and mixtures thereof. Other suitable metals include cobalt-chromium, stainless steel, nickel and nickel alloy, zirconium and niobium. In a preferred embodiment, the metal particles are made of titanium.

The metal hydride particles are composed of titanium hydride, tantalum hydride, etc. Preferably the hydride is based on the same metal as the metal used to obtain the body. Metal hydrides are commercially available, usually in the form of a powder, having a particle size of about 20-120 μm. To assist the sintering, the amount of metal hydride employed is about 5-10 wt. %, based on the weight of the porous body. To assist the pyrolysis the same amounts may be used, based on the weight of metal particles present in impregnated foam.

Apart from the binder, other additives may be used. These additives comprise deflocculants, such as Dolapix™.

Furthermore, viscosity modifying agents may be used, to control the viscosity of the slurry. Preferably the viscosity of the slurry is from 4000 cP to 12000 cP, as measured on a Brookfield viscometer, using a HA5 spindle at a spindle speed of 20 rpm.

As a further additive, pH-modifying agents, such as ammonia may be employed to control the surface charge of the titanium material.

Average particle size and particle size distribution of the metal particles are important parameters in preparing the articles of the present invention. Generally, the sintering of fine powders is easier than the sintering of coarse powders. For this reason, fine powder with diameter smaller than 5 μm would be desirable, but are however, difficult to obtain commercially. Particles larger than about 120 μm tend to segregate in the slurry and may hamper the formation of a homogeneous suspension. Preferred average particle sizes for the metal are from 5-100 μm, even more preferably from 10-50 μm. Metal particles which are commercially readily available have a particle size of 325 mesh (44 μm).

Polyurethane (PU) foam is a very suitable polymeric material to be used according to the present invention, since it has an excellent pore structure. Preferably, PU foam having a pore size of 500-2000 μm is used. Although other polymers, such as polymethyl methacrylate, polyether, polyester, and mixtures thereof may be used as well, these polymers are less suitable, because it was found that these polymers do not pyrolyze as well as PU and/or have a less advantageous pore structure.

After preparing the slurry was, the polymeric foams are contacted with the slurry, so that the foam becomes soaked with slurry. Excessive slurry may be removed, e.g. by applying pressure by squeezing. Subsequently, the slurry-loaded foams may be dried, typically at 50-150° C. After a suitable period of time of drying at elevated temperature, e.g. 1-5 hours, the sample may be further dried at room temperature, e.g. for 1-2 days.

In case a coating is to be prepared, the metal implant is preferably carefully cleaned with degreasers, detergents, or solvents and rinsed with water. A thin layer of slurry may then be applied onto the substrate surface by dipping into the slurry or painting with the slurry. The slurry-loaded foam is then applied onto the surface of the substrate to be coated. It will be drying is carried out at the above-mentioned temperatures and at pressures of about 0.001-0.1 mbars.

After drying, the sample is subjected to pyrolysis, in order to remove the polymeric foam and binder (and other organic or pyrolysable material, if present) from the sample to yield a porous body or coating of metal particles. The removal of binders and foam is performed through heat processing under a non-oxidative atmosphere. During the heat processing of porous titanium, the rate of removal of binder and PU is an important parameter. Evaporating the binder too fast, may cause “blisters” to form, while evaporating the binder too slow may causes parts of the sample to collapse. Pyrolysis is preferably carried out under vacuum or reduced pressure conditions, typically 10⁻¹ to 10⁻⁶ mbars and preferably at about 10⁻²-10⁻³ mbars. The pyrolysis is preferably carried out at a temperature from about 50-650° C., and even more preferably at about 150-550° C. Preferred time periods for removing the binders and foam range from about 8 to 72 hours, even more preferably from about 12 to 16 hours.

After removal of binders and foams and optionally other material by the pyrolysis step, the resulting body, or coated substrate is ready for final sintering, if desired. The sintering may be performed in one or multiple steps. It is preferred that the sintering is carried out at a temperature of about 700-1500° C., preferably for about 10-26 hours. More preferably the sintering is carried out at a temperature of about 800-1400° C., preferably for about 12-18hours. The sintering atmosphere is a non-oxidation atmosphere, proceeding, for example, in argon or other inactive gases, under a vacuum or reduced pressure conditions, about 10⁻³ to 10⁻⁶ mbars.

It is noted that suitable durations for the respective drying, pyrolysis and sintering steps, depend on the size of the foam materials and may vary accordingly, the above-mentioned preferred values for the these durations being given for a typical sample size of several cm. Depending on the specific case, each of the drying, pyrolysis and sintering step is generally carried out in a period of time ranging from several hours to several days.

In order to prepare articles, or coat substrates, that may be used as implants, the foam may be formed into the desired shape and size, e.g. by cutting, after which the method of the invention is carried out to produce a sintered metal body, or sintered coated metal substrate. A dimensional shrinkage of 5-10% will normally occur in the drying and sintering stage, which may be corrected for in cutting the foam that is used as starting material. The sintered metal body or coating may be further machined with usual means, such as drilling, milling, etc., to give it its desired shape and size.

According to the method of the invention, it is possible to produce articles or coatings that have a porous metal structure with a porosity of at least 50%, having a mean pore size of at least 400 μm, wherein the pores are interconnected. The porous metal articles of the invention have a compressive strength ranging from 5 MPa up to 40 MPa, or even higher. Strength is obviously related to porosity. In the case of 80% porous titanium alloy, a compressive strength of 10 MPa or higher may be obtained in accordance with the invention, which is suitable for applications in implants. Typically, 50-90% porous implants can be provided, having a compressive strength ranging from 5-40 MPa. The mechanical compressive strength which may be obtained in accordance with the present invention is sufficient for load-bearing purposes. In case high strengths are desired, one could choose bulk metal implants with superior mechanical properties on which a porous metal coated is applied. This unique combination will ensure biological fixation of implants to skeleton via bone growth into the porous metal and transfer of physiological loads and mechanical forces from bone to implants. The porous coated structure applied onto a bulk metal implants will increase primary fixation of orthopedic or dental prostheses as well as transfer of biomechanical forces.

Articles or coated substrates according to the invention are therefore particularly suitable for use as an implant, such as bone replacement material or scaffolds (viz. porous structures to which living tissue may be applied in vitro and which are subsequently implanted). With respect to a coating according to the invention, it is noted that this coating is particularly beneficial when applied to such an area of e.g. a hip implant to achieve proximal fixation, and no distal fixation. The thickness of the coating is preferably 2-3 layers of pores, such as 1-5 mm depending on the pore size and application of the coated substrate. If desired, a ceramic coating, such as a calcium phosphate coating may be applied onto the porous metal body or coating.

The invention will now be illustrated by the following examples.

### EXAMPLE 1

Titanium powder containing particles having an irregular shape and an average particle size of 325 mesh (<44 μm) was obtained from the Beijing Non-Ferrous Institute in China. The chemical composition of the powder was as follows:

Element

| | N | H | O | C | Fe | Ti |
|---|---|---|---|---|---|---|
| W/w % | 0.06 | 0.06 | 0.5 | 0.05 | 0.15 | balance |

A slurry was prepared by mixing the titanium powder, with a 25% ammonia solution (Merck), Dolapix (Zschimmer & Schwarz Gmbh, Germany) and methylcellulose (Dow U.S.A) in the amounts given in Table 1 under stirring. Stirring was continued until homogeneous slurry was obtained.

TABLE 1
Composition of titanium slurry for Example 1

| Ingredient | Quantity (g) | Wt. % |
|---|---|---|
| Demi water | 100 | 30 |
| Dolapix CE64 | 4 | 1.2 |
| Ammonia (25%) | 7 | 2.2 |
| Methylcellulose | 2 | 0.6 |
| CMC | 0.46 | 0.15 |
| Ti powder | 222 | 65 |
| Total | 333 | |

Polyurethane foam was soaked in the slurry and squeezed by hand to remove slurry. After drying, the sample was placed in a vaccum furnace on top of 16 g of titanium hybride (obtained from RaoTai China), the titanium hybride being present on the bottom of the furnanc. The furnance was set to follow a present temperature and pressure program. The temperature program comprised heating the impregnated foam to remove binders and the foam during about 1000 minutes during which the temperature increased from 25 to about 350° C. The pyrolysis was carried out at pressure of .001 mbars. Directly following the sintering the heating was stopped and the pressure was normalized.

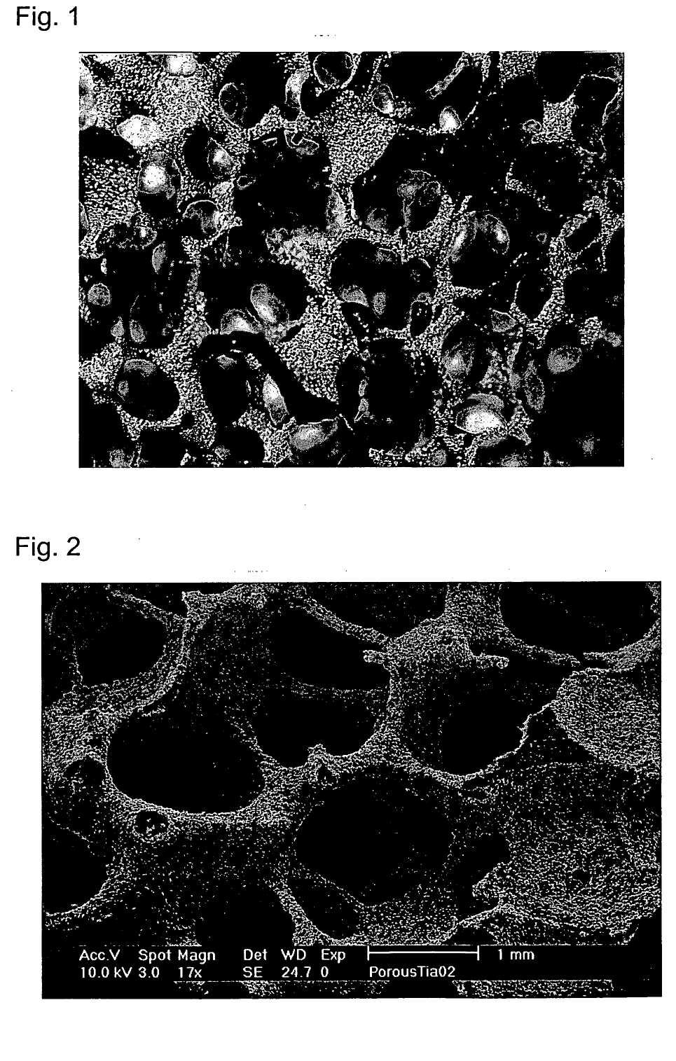

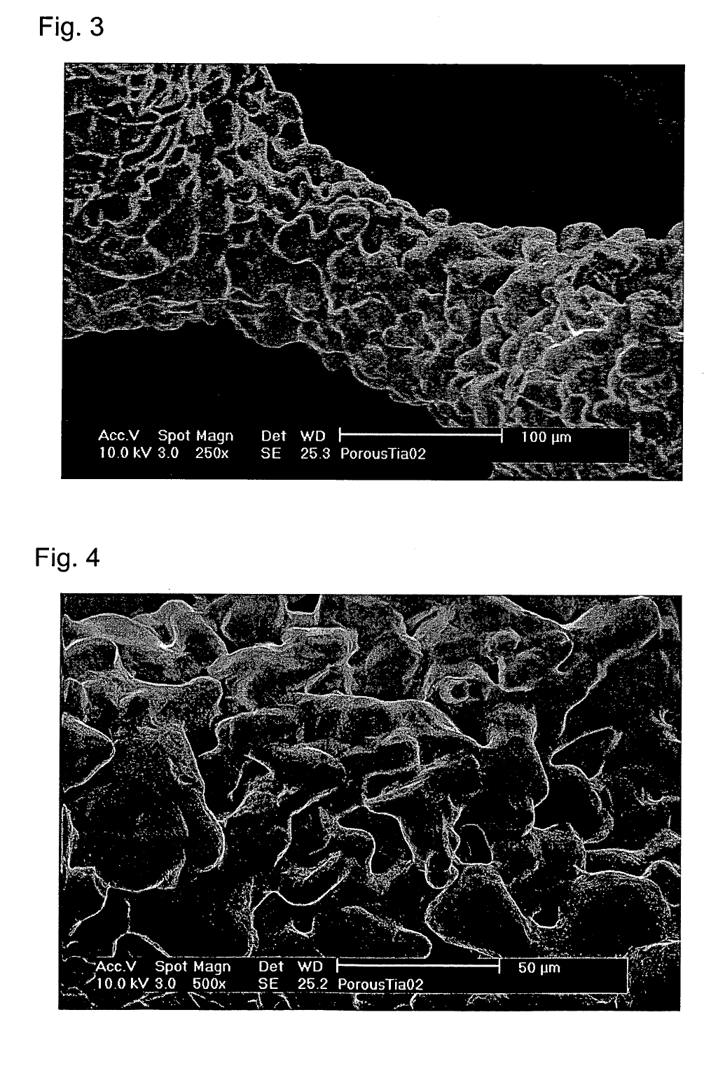

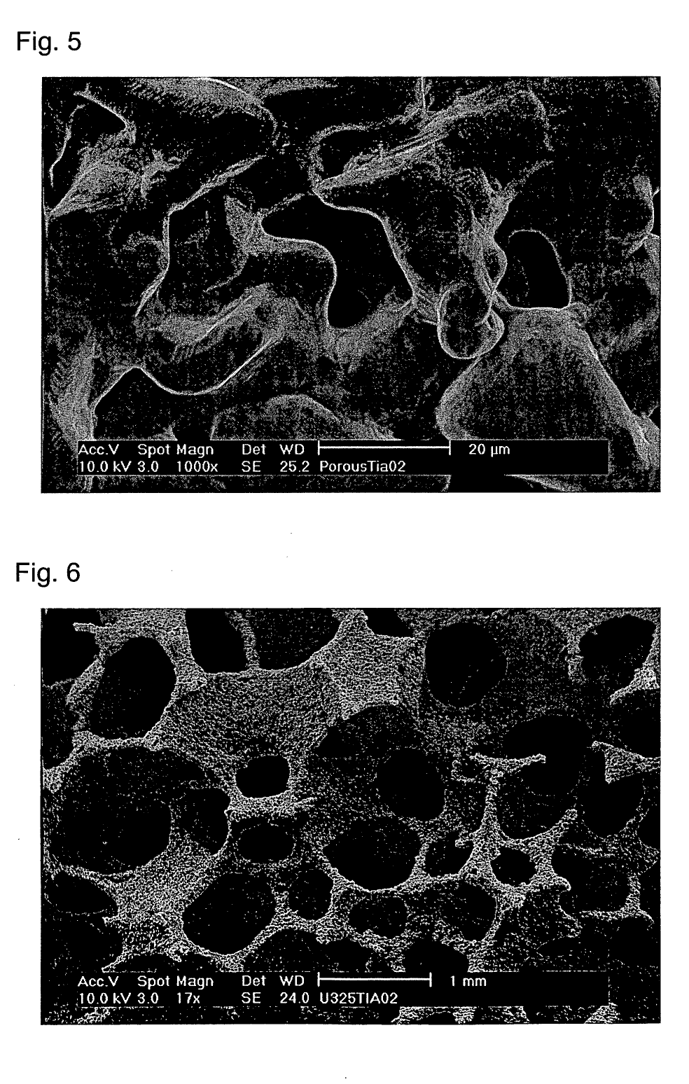

Obtained porous titanium, microscopic photographs were taken as shown in FIGS. 1-5. FIG. 1 shows the structure under an optical microscope with a magnification of 20×. FIG. 2 shows the structure of porous titanium under SEM, FIG. 3 shows the strut of porous titanium. FIG. 4 shows microstructure at a magnification 20×,and FIG. 5 shows the same microstructure at a high magnification of 1000×. The pictures show a interconnected system of regularly shaped pores.

### EXAMPLE 2

Titanium alloy powder having an spherical shape and an average particle size of 325 mesh (<44 μm) was obtained from the Northwest Non-ferrous Institute in China. The chaemical composition of the powder was as follows:

| Ele- | | | | | | | | |
|---|---|---|---|---|---|---|---|---|
| ment | N | H | O | C | Fe | Al | V | Ti |
| W/w % | 0.05 | 0.015 | 0.2 | 0.08 | 0.3 | 5.5-6.5 | 3.5-4.5 | balance |

A slurry was prepared by mixing the titinium alloy powder, with a 25% ammonia solution (Merck), Dolapix (Zschimmer & Schwarz Gmbh, Germany), PEG4000 (Merck) and Carboxymethylcellulose (Merck) in the amounts given in Table 2 under stirring. Stirring was continued until homogeneous slurry was obtained.

TABLE 2
Composition of titanium slurry for Example 2

| Ingredient | Quantity | Wt. % |
|---|---|---|
| Demi water | 100 | 25 |
| PEG4000 | 28 | 7 |
| Dolapix | 6 | 1.5 |
| Ammonia | 5.2 | 1.3 |
| CMC | 0.8 | 0.20 |
| Ti powder | 264 | 66 |
| Total | 404 | |

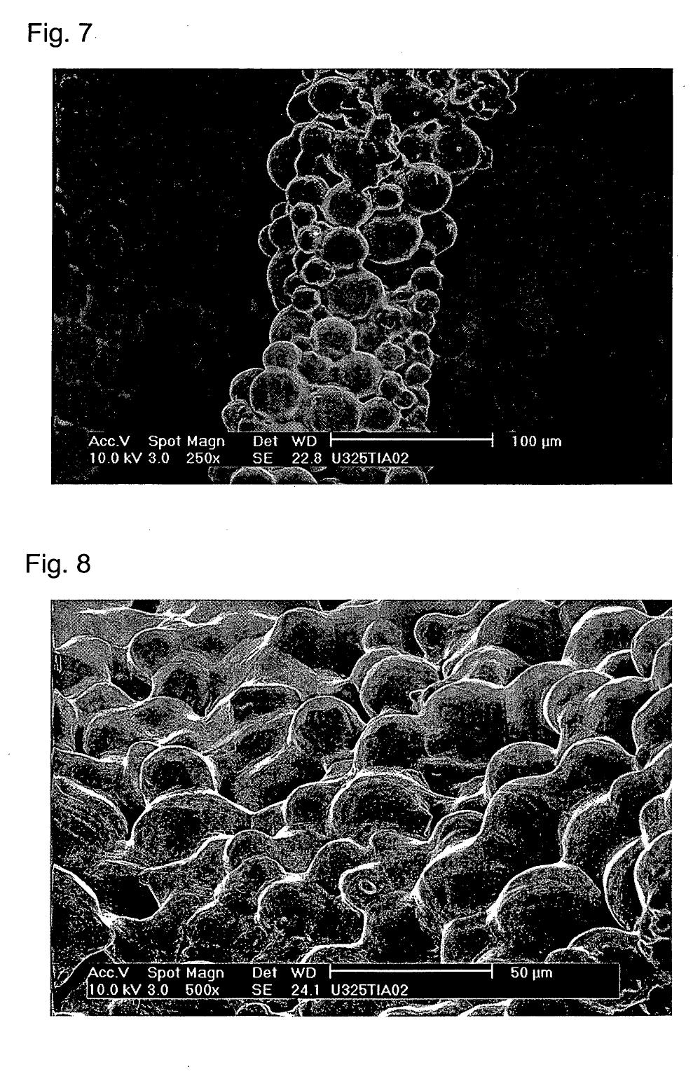

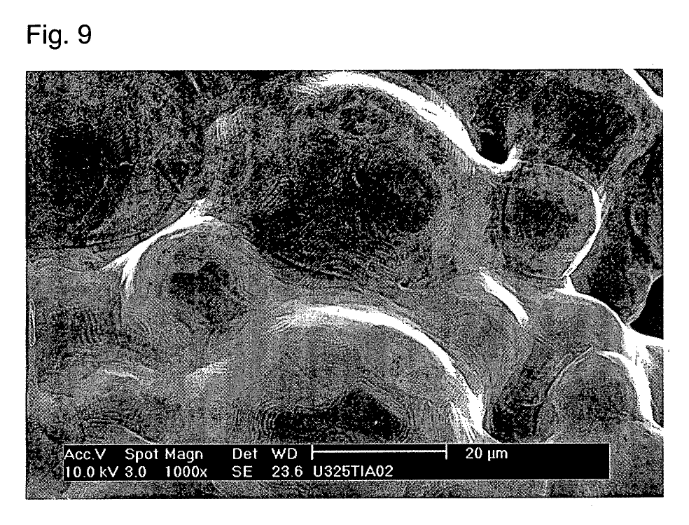

Using this slurry, the procedure of Example 1 was repeated. Of the obtained porous titanium, microscopic photographs were taken as shown in FIGS. 6-9. FIG. 6 shows the structure of porous titanium under SEM. FIG. 7 shows the strut of porous titanium, FIG. 8 shows microstructure at a magnification of 500×, and FIG. 9 shows the same microstructure at a higher magnification (1000×).

Again the pictures show a interconnected system of regularly shaped pores.

The porous structures obtained in both Example 1 and 2 had a mechanical compressive strength of 10 MPa (as measured on a Hounsfield test bench at 1 mm/min), which is sufficient for load bearing purposes in implant applications.

### EXAMPLE 3

In this example, methods and techniques to apply a porous metal coating onto a metal implant are given. Titanium alloy (Ti6A14V) plates of 20×20×1 mm are used. The Ti6A14V plates are carefully cleaned in acetone 15 minutes, then in 70% ethanol 15 minutes, finally in demineralised water 15 minutes.

A titanium slurry is prepared as previously described in examples 1 and 2. The Ti6A14V plates are dipped into the titanium slurry and then dried at 80° C. for 30 minutes. The titanium slurry can also be painted onto the Ti6A14V plates. To ensure a good coverage of Ti6A14V plates, the cycle of dipping-squeezing can be repeated several times, in practice 2-3 times for a uniform film of reactive titanium applied onto the Ti6A14V plates.

Polymeric sponge made of polyurethane (PU) is selected for optimal porosity and pore size. PU foams (Recticel) having 30 pore cells per inch (R30) or pore size of 1200 microns are used. The PU foam needs to be cut into the shape as design. It should be taken into consideration that 3-5% dimensional shrinkage will occur in the drying and sintering stage. The PU foam is cut to suitable dimensions (i.e. 25×25×7 mm) using a blade or any other cutting device. The PU foam is then dipped into the metal slurry and dried at 80° C. for 30 minutes. The dipping-squeezing process is repeated until all the struts of the PU foam are evenly coated with Ti(alloy) slurry.

The PU foam covered with the titanium slurry is applied onto the Ti6A14V plate. The substrate Ti6A14V plates are painted with the titanium slurry and then contacted with titanium slurry impregnated PU foams and finally the assembly plate/foam is dried at 80° C. for 30 minutes. After drying, the samples are placed in a vacuum furnace on top of titanium hydride powder. The furnace was set to follow a preset temperature and pressure program. The temperature program comprised heating the impregnated foam to remove binders and the foam during about 1000 minutes during which the temperature increased from 25 to about 350° C. The pyrolysis was carried out at a pressure of 0.01 mbars. Directly following the removal of the binder, the temperature was raised to 1350° C. and the product was sintered at this temperature during about 140 minutes. The sintering was carried out at a pressure of 0.00002 mbars. Following the sintering the heating was stopped and the pressure was normalized.

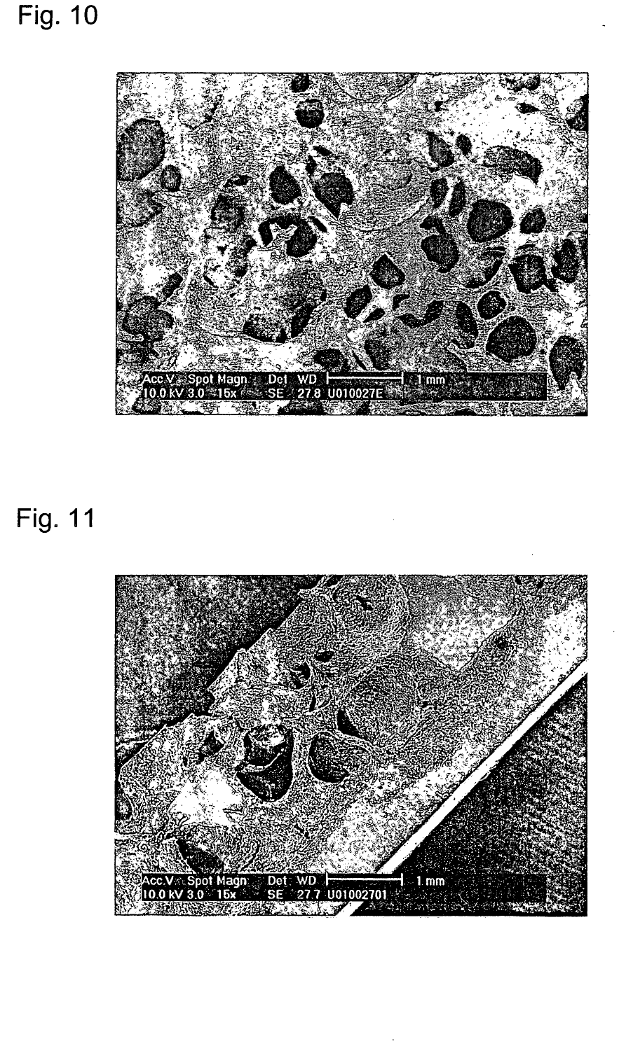

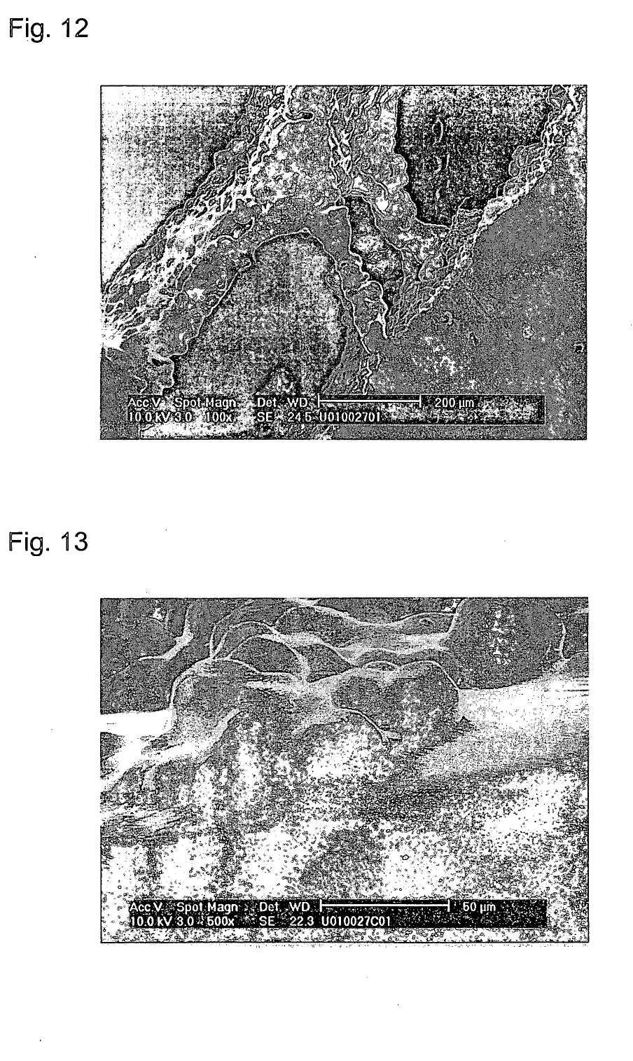

Of the obtained porous titanium coating, microscopic photographs were taken as shown in FIGS. 10-13. FIG. 10 shows the structure of the porous coated layer under SEM. FIG. 11 shows a cross-section of the porous coated layer. FIG. 12 shows the strut of porous titanium, and FIG. 13 shows the diffusion of particles to the substrate.

### EXAMPLE 4

Porous titanium alloy cylinders were tested under compressive load. Porous titanium alloy cylinders of 8 mm in diameter and 5-11 mm in thickness were placed in a single axis mechanical test bench (Zwick/Z050, Germany) with a 50 kN load cell. A crosshead speed of 1 mm/min was applied. The load-strain curve was recorded. The mean value and standard deviation of compressive strength is 10.32±3.1 Mpa.

TABLE 3
Mechanical properties

| | Compressive |
|---|---|
| Size | strength(MPa) |
| Ø7.9 × 5 | 8.89 |
| Ø7.9 × 5 | 14.41 |
| Ø7.9 × 5 | 15.5 |
| Ø7.9 × 5 | 13.54 |
| Ø7.9 × 6.7 | 10.96 |
| Ø7.9 × 6.7 | 11.33 |
| Ø7.9 × 6.7 | 8.04 |
| Ø7.9 × 6.7 | 7.06 |
| Ø7.9 × 11.6 | 7.02 |
| Ø7.9 × 11.6 | 9.7 |
| Ø7.9 × 11.6 | 7.02 |
| Average | 10.32 ± 3.1 |

### EXAMPLE 5

Biocompatibility and Soft Tissue Ingrowth in Rats

This example gives results of an animal study with interconnected porous Ti6A14V implants. The porous titanium bodies were implanted subcutaneous in rats for 1, 2, and 4 weeks and histology has been performed. Eighteen male wistar rats, weight 150-200 grams, were used for this experiment. Each rat received 2 implants two bare porous Titanium implanted under skin in each side of the spine. On each site of the spinal core two lateral incisions of 2 cm were made. Using a blunt scissor created two subcutaneous pockets and implants were placed in the subcutaneous pockets.

Data Analyses

After 1, 2 and 4 weeks, the rats were sacrificed, the implants with surrounding tissue were explanted and were stored in karnovsky's reagens at 4° C. The retrieved implants were washed in phosphate buffer solution, dehydrated in series of ethanol 70%-100%. The implants were transferred to methylmethacrylate, which polymerized at 37° C. for a week. Histological sections were made longitudinal implants with a thickness of 10-15 μm on a diamond saw. The porous titanium implants were stained with 1% methylene blue and 0.3% basic fuchsin and exanimate with the light microscopy.

Results

Light Microscopical Evaluation

Porous Titanium (Ti6A14V)

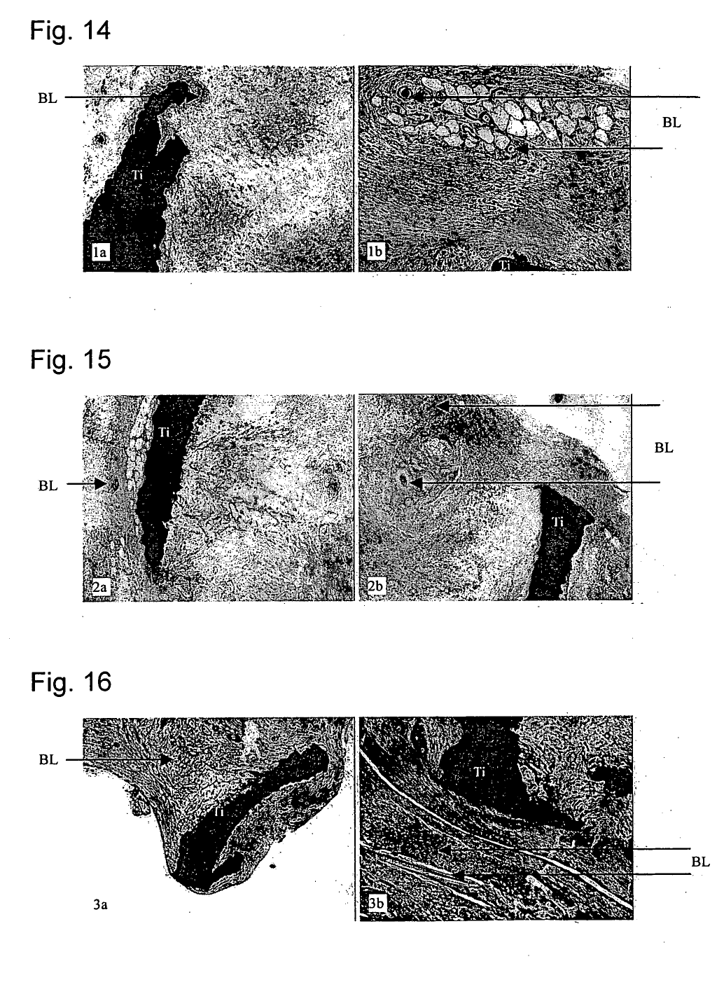

After one-week implantation, light microscopical evaluation showed no adverse tissue reaction nor giant cells and macrophages. An intervening fibrous tissue encapsulated the porous titanium implants. In the inner part of the porous titanium implants fibroblasts and fibrocytes were observed. However, on some places no tissue could be found, which suggest that the encapsulation of the intervening fibrous tissue is not complete. On the borders of the porous titanium implants, connective tissue was seen. Connective tissue consists of several different tissues, like fat cells, looseness connective tissue, and unorganized/organized connective tissue. After one-week implantation, namely unorganized connective tissue and fat cells were found. Further, blood vessels appear in the connective tissue, which suggest a vascular growth within the porous implants (see FIGS. 14, 15, and 16).

After two weeks implantation, no macrophages cells were found near the porous titanium implant. Further encapsulation of the porous titanium implant was complete, no empty space was observed. Thereby, fibroblast and fibrocytes were seen in the middle part of the implant. After two weeks implantation, looseness and unorganized connective tissue was found on the borders of the porous titanium implant. Further blood vessels were observed in the surrounding tissue.

After four weeks of implantation, a thicker encapsulation by fibrous tissue than after one and two weeks was observed. Furthermore, the connective tissue was more organized and more blood vessels were observed, which suggest that a better vascularity was achieved during the implantation time and no adverse reaction was found. Also blood vessels were observed in the middle part of the porous titanium implant than compared to the one and two weeks implantation.

In summary, the porous titanium alloy bodies showed good biocompatibity with soft tissue and a normal fibrous tissue encapsulation. Tissue, blood vessels as well as fibroblast cells were found in the pores of the porous titanium implants.

1. Method for preparing a porous body, suitable for the production of a porous metal article, comprising the steps of providing a polymeric foam, which foam is impregnated with a slurry of metal particles, drying the impregnated foam, followed by pyrolysis in the presence of metal hydride particles.

2. Method according to claim 1, further comprising sintering of the porous body, which sintering is carried out in the presence of metal hydride particles.

3. Method for providing a porous metal coating to a metal substrate comprising the steps of providing a polymeric foam, which foam is impregnated with a slurry of metal particles, pasting the impregnated foam onto the substrate, drying the impregnated foam, followed by pyrolysis in the presence of metal hydride particles, and sintering.

4. Method according to claim 3, wherein the substrate comprises a metal selected from titanium, tantalum, titanium alloy, tantalum alloy, cobalt-chromium, stainless steel, nickel and nickel alloy, zirconium, niobium and mixtures thereof.

5. Method according to claim 4, wherein the substrate comprises titanium or a titanium alloy.

6. Method according to any of the previous claims, wherein the presence of said metal hydride particles is provided by placing metal hydride particles in the environment without contacting said impregnated foam in which said pyrolysis or said sintering is carried out.

7. Method according to any of the previous claims, wherein said metal is selected from titanium, tantalum, titanium alloy, tantalum alloy, cobalt-chromium, stainless steel, nickel and nickel alloy, zirconium, niobium and mixtures thereof.

8. Method according to claim 7, wherein said metal is titanium or a titanium alloy.

9. Method according to any of the previous claims, wherein said metal hydride is based on the same metal as said metal particles.

10. Method according to any of the previous claims, wherein said polymeric foam comprises polyurethane.

11. Method according to any of the previous claims, wherein said slurry further comprises one or more of the following additives: a binder, a defloculant, a viscosity modifying agent and/or a pH-modifying agent.

12. Method according to claim 11, wherein said slurry comprises a binder selected from PEG4000, methylcellulose and/or carboxyl methyl cellulose (CMC).

13. Method according to any of the previous claims, wherein said metal particles have a mean diameter of 5-100 μm.

14. Method according to any of the previous claims, wherein said pyrolysis is carried out at a pressure of 10⁻³-10⁻² mbars.

15. Method according to any of the previous claims, wherein said sintering is carried out at a pressure of 10⁻⁶-10⁻⁴ mbars.

16. Method according to any of the previous claims, wherein said pyrolysis is carried out at a temperature of 150 to 550° C.

17. Method according to any of the previous claims, wherein said sintering is carried out at a temperature of 1050-1350° C.

18. Article of manufacture comprising a porous body obtainable by a method according to any of the claims 1, 2 or 4-17.

19. Article of manufacture comprising a coated substrate obtainable by a method according to any of the claims 3-17.

20. Article according to claim 18 or 19, which is a medical implant, preferably a bone replacement material or a scaffold.

21. Medical implant comprising a porous metal structure or coating with a porosity of at least 50%, having a mean pore size of at least 400 μm, wherein the pores are interconnected, which implant has a compressive strength of at least 10 MPa, wherein the metal is selected from titanium, tantalum, titanium alloys, tantalum alloys and combinations thereof.

22. Use of a metal hydride in a sintering and/or pyrolysis process for the manufacture of porous metal articles from metal particles.
